(19) United States
(12) Patent Application Publication
(10) Pub. No.: US 2005/0272930 A1
(43) Pub. Date: Dec. 8, 2005

(54) Process for the prepation of 3-hydroxy-(2-thienyl)propanamines

(76) Inventors: William Hems, Ely (GB); Kai Rossen, Hanau (DE); Dietmar Reichert, Eschau (DE); Klaus Kohler, Hainburg (DE); Juan Jose Almena Perea, Hanau (DE)

Correspondence Address: FITCH, EVEN, TABIN & FLANNERY, P. O. BOX 65973, WASHINGTON, DC, 20035, US

(21) Appl. No.: 10/521,799

(22) PCT Filed: Jul. 21, 2003
(86) PCT No.: PCT/EP03/07927

§ 371 Date: Jan. 21, 2005

(30) Foreign Application Priority Data
Jul. 24, 2002 (DE) 102 33 724.1
Dec. 11, 2002 (DE) 102 58 098.7

(51) Int. Cl. C07D 413/12; C07D 333/22

(52) U.S. Cl. 544/97; 549/76

(57) ABSTRACT

The present invention relates to a process for the preparation of compounds of the general formula (I) 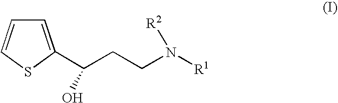 by catalytic enantioselective hydrogenation of the corresponding ketones. There are used inter alia ruthenium catalysts with chiral diamines and chiral biphosphines as ligands.

The present invention is directed to a process for the enantioselective hydrogenation of special α-heteroaryl ketones. In particular the invention relates to a process for the preparation of compounds of the general formula (I):

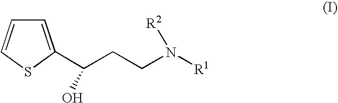

This class of compounds is used as intermediates for the synthesis of enantiomer-pure bioactive substances, e.g. Duloxetine®.

Duloxetine®, (S)-(+)-N-methyl-3-(l-naphthyloxy)-3-(2-thienyl)propanamine hydrochloride, is a pharmaceutical that is used as an antidepressant and for the treatment of urinary incontinence. It inhibits the reuptake of both norepinephrine (?) and serotonin. The synthesis of Duloxetine® is described in detail in EP-A-273 658, EP-A-457 559 and EP-A-650 965.

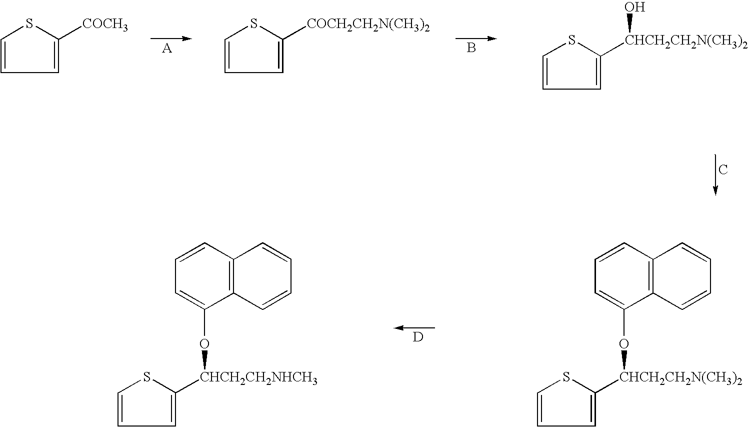

Starting from 2-acetylthiophene, in stage A an aminomethylation is carried out with dimethylamine and formaldehyde (Mannich reaction). The 3-dimethylamino-1-(2-thienyl)-1-propanone that is formed is reduced in step B by means of complex hydrides to the corresponding alcohol 1-hydroxy-1-(2-thienyl)-3-dimethylaminopropane. The alcohol is then converted in step C with an alkali metal hydride and 1-fluoronaphathalene, optionally in the presence of a potassium compound (see EP-A-650 965), into the naphthyl derivate N,N-dimethyl-3-(1-naphthyloxy)-3-(2-thienyl)-propanamine. In the last step D the amino group is then demethylated by reaction with a chloroformic acid ester, preferably phenyl chloroformate or trichloroethyl chloroformate, optionally in the presence of a mixture of zinc and formic acid (EP-A-457 559), followed by alkaline hydrolysis of the carbamate to form N-methyl-3-(1-naphthyloxy)-3-(2-thienyl)propanamine. The (S)-(+)-enantiomer of the product in the hydrochloride form is the desired compound Duloxetine®.

Since a racemate is usually formed in the above synthesis of N-methyl-3-(1-naphthyloxy)-3-(2-thienyl)propanamine, special measures are necessary for the selective preparation of the (S)-(+)-enantiomer. For example, EP-A-457 559 discloses an asymmetric reduction in step B by a complex of lithium aluminium hydride and a chiral ligand.

The disadvantage with the aforementioned synthesis pathway is in particular step D, i.e. the demethylation. In this connection highly corrosive chloroformic acid esters, optionally in combination with toxic zinc, are used in the last stage of the synthesis of a medicament, and carcinogenic methyl chloride is released. Complicated separation and purification steps consequently have to be subsequently employed. A conversion of the dimethylamino group into the desired monomethylamino group in an earlier synthesis stage would therefore be desirable. An alternative synthesis pathway for Duloxetine® would lead via the conversion of (S)-N-methyl-3-hydroxy-3-(2-thienyl)propanamine to (S)-(+)-N-methyl-3-(1-naphthyloxy)-3-(2-thienyl)propanamine in the last step.

In EP-A-457 559 the enantioselective reduction of N-benzyl-N-methyl-1-(2-thienyl)-1-propanone to N-benzyl-N-methyl-3-(β-hydroxy)-3-(2-thienyl)propanamine is described in Example 1B. However, there is no indication of how N-methyl-N-benzyl-3-(β-hydroxy)-3-(2-thienyl)propanamine can be debenzylated. Investigations carried out by the inventors of the present application have shown that the conversion of N-methyl-N-benzyl-3-hydroxy-3-(2-thienyl)propanamine with hydrogen in the presence of conventional palladium catalysts in solvents such as alcohols and acetic acid does not lead to the desired debenzylated monomethylamine N-methyl-3-hydroxy-3-(2-thienyl)propanamine.

Catalytic enantioselective hydrogenations of C═O double bonds have in the meantime become standard reactions in organic chemistry. For example GB2351735 discloses the use of certain catalysts in the reduction of special α-aryl methyl ketones. Reference is also made to the use of so-called diphosphine ligands in combination with ruthenium and a chiral diamine in the reduction of this substrate.

It has been found however that one specific catalyst or a class of catalysts cannot be used equally well in all hydrogenations, but that each reduction problem has to be investigated separately with regard to the catalyst use and the conditions. This is all the more so in the case of hydrogenations that take place with catalysts that consist not only of a ligand and a transition metal but that, as outlined in the above case, require two different ligands and the transition metal in order to be sufficiently active.

The object of the present invention was to provide a process for the enantioselective reduction of special α-heteroaryl ketones. This process should operate particularly well on an industrial scale having regard to economic and ecological aspects, i.e. should be superior to conventional methods of the prior art as regards space-time yield, enantiomer excesses, robustness and raw material costs or waste disposal costs. In particular the process should be suitable for providing in an advantageous manner specific enantiomer-enriched alcohols as intermediates for the preparation of Duloxetine®.

This object is achieved according to the claims. Claim 1 is directed to the process according to the invention. Dependent subclaims describe preferred embodiments. Claim x is directed to a specific intermediate product formed in the present reduction.

Accordingly, in a process for the preparation of enantiomer-enriched compounds of the general formula (I) 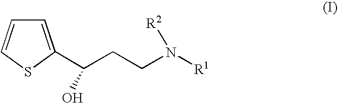
wherein

R¹ and R² independently of one another denote H, (C₁-C₈)-alkyl, (C₁-C₈)-acyl, (C₁-C₈)-alkoxycarbonyl, (C₃-C₈)-cycloalkyl, (C₆-C₁₈)-aryl, (C₇-C₁₉)-aralkyl, (C₃-C₁₈)-heteroaryl, (C₄-C₁₉)-heteroaralkyl, ((C₁-C₈)-alkyl)₁₋₃-(C₃-C₈)-cycloalkyl, ((C₁-C₈)-alkyl)₁₋₃—(C₆-C₁₈)-aryl, ((C₁-C₈)-alkyl)₁-3—(C₃-C₁₈)-heteroaryl,

or the radicals R¹ and R² together form a (C₁-C₈)-alkylene bridge, wherein these may be substituted with one or more (C₁-C₈)-alkyl, (C₃-C₈)-cycloalkyl, (C₆-C₈)-aryl, (C₇-C₁₉)-aralkyl, (C₃-C18)-heteroaryl, (C₄-C₁₉)-heteroaralkyl radicals with the formation of further chirality centres, by enantioselective hydrogenation of compounds of the general formula (II) 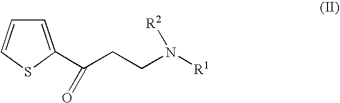
wherein R¹ and R² have the meanings given above, the aforementioned object is achieved especially advantageously according to the invention in a particularly surprising and in no way foreseeable manner by using for the hydrogenation a catalyst comprising an enantiomer-enriched bidentate phosphorus-containing ligand, a transition metal and a diamine, preferably a chiral diamine. Enantiomer-enriched alcohols of the general formula (I) can be prepared with the aid of these measures in very short reaction times and with high yields as well as excellent enantiomer excesses. It is particularly advantageous if in the above reaction compounds are used in which R² denotes a COR¹ group.

The term phosphorus-containing ligands is understood by the person skilled in the art to mean preferably bidentate biphosphines or biphosphites, or their mixed forms. Phosphite-containing ligands that may advantageously be used are described for example in J. Am Chem. Soc. 1994, 116, 4101; J. Org. Chem. 1997, 62, 6012; Asymmetry 10 (1999), 2129-2137; Asymmetry.10 (1999), 4009 or also in the supplement “Catalytic asymmetric synthesis”, Iwao Ojima, Second Edition, Wiley-VCH 2000 and the literature cited therein. As biphosphine ligands there may be used the ligands mentioned in “Catalytic asymmetric synthesis”, Iwao Ojima, Second Edition, Wiley-VCH 2000. A further summary is published in ACS Symposium Series 641 “Reductions in Organic Synthesis, Chapter 2: Chiral Ruthenium(II)catalysts for Asymmetric Hydrogenation”, 1996. An advantageous selection is shown in the following Scheme 1.

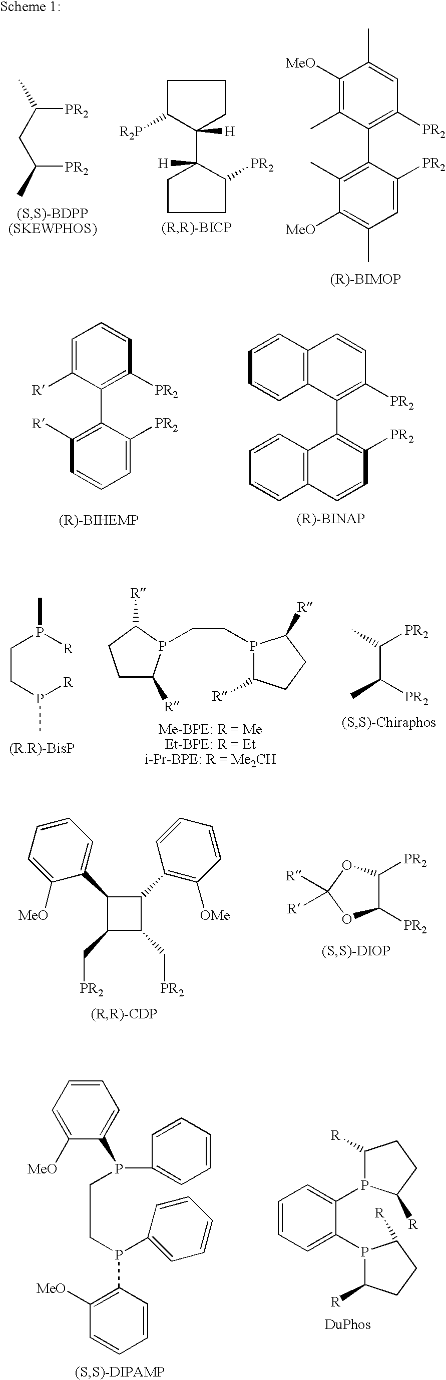

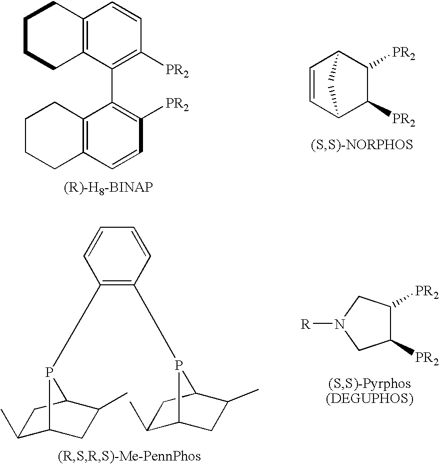

Further suitable compounds are shown in Scheme 2.

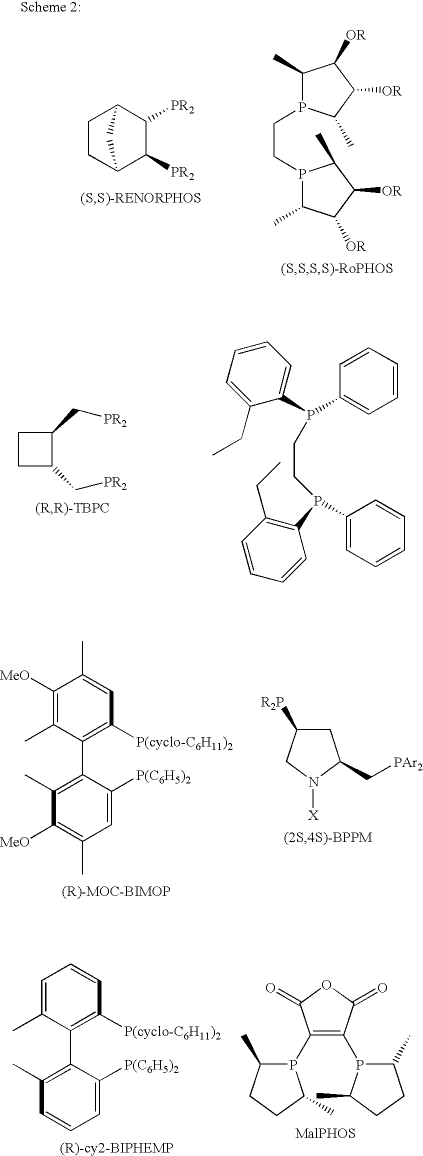

It is particularly advantageous to use chiral phosphorus-containing ligands selected from the group consisting of Deguphos, Binap, Phanephos, Norphos, DIOP, Duphos, Prophos, BDPP, BPPM, Malphos, Rophos or Basphos as described in Angew. Chem. 2001, 113, 40-75 and the literature cited therein; in J. Org. Chem. 1999, 64, 6907; in Synthesis 1997, 9, 983-1006 or in Org. Lett. Vol. 2, No. 12, 2000. The compounds disclosed in DE10100971 may also be used equally well.

Particularly suitable as phosphite ligands are the ligands shown in Scheme 3.

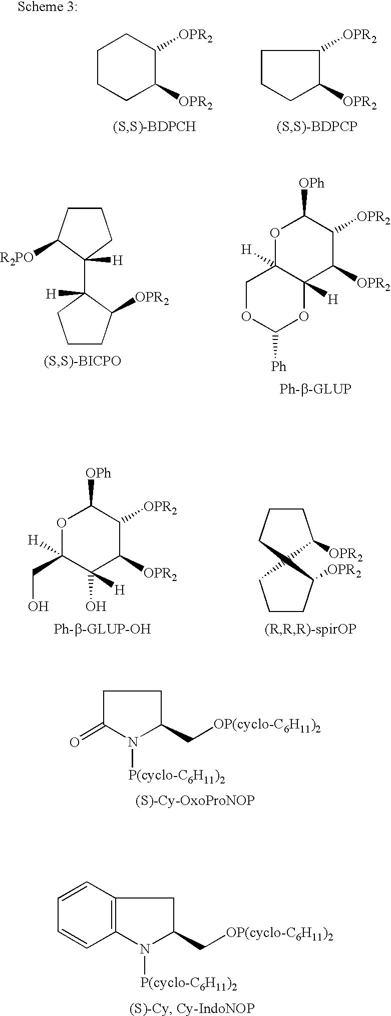

As diamine there may in principle be used all chiral 1,2-diamine species that exhibit a sufficient activity or selectivity in the catalyst under consideration. Suitable diamines are in particular those mentioned in “Catalytic asymmetric synthesis”, Iwao Ojima, Second Edition, Wiley-VCH 2000. A selection is shown in the following Scheme 4.

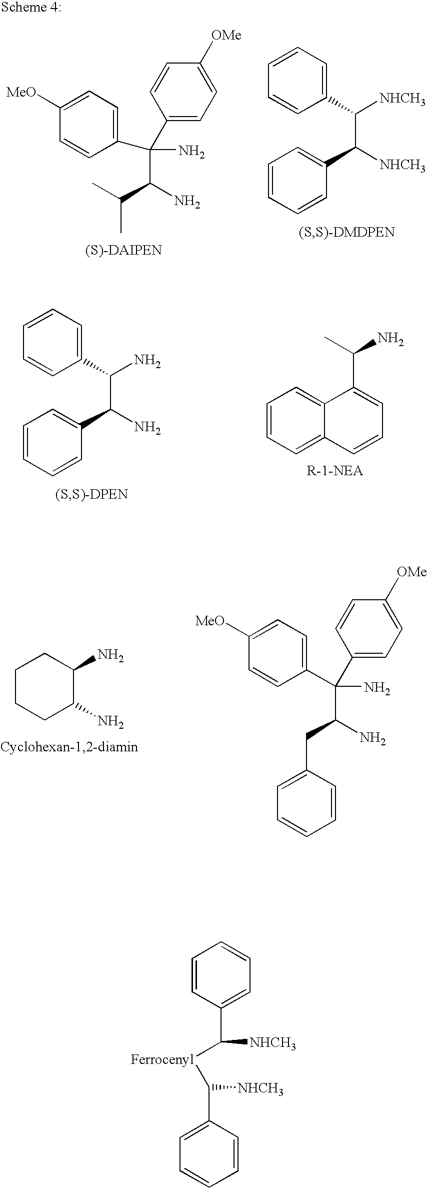

The use of chiral compounds selected from the group DAIPEN, DPEN, DMDPEN, 1,2-cyclohexyldiamine has proved particularly advantageous.

As transition metals there may in principle be used all transition metals that appear to the person skilled in the art to be suitable for the specific hydrogenation problem. In particular transition metals are selected from the group consisting of Ru, Rh, Ir, Pd, in any oxidation state that appears suitable for this purpose. Various counterions such as for example OTF⁻, ClO₄⁻, SbF₆⁻, PF₆⁻ or BF₄⁻ or the like may be mixed for the purposes of charge equalisation with the overall complex of diamine, phosphine ligand and transition metal.

The advantageous catalyst resulting therefrom has the following structure V:

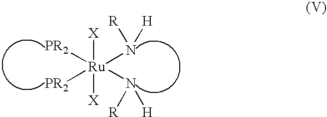

X is an anion as specified above, for achieving electrical neutrality. Preferred ligands of the general formula (VI) 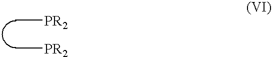
have as substituents R a (C₃-C₈)-cycloalkyl, (C₆-C₁₈)-aryl, (C₇-C₁₉) -aralkyl, methoxy- (C₇-C₁₉) -aralkyl group, wherein the phosphane or phosphite groups are covalently bonded to a chiral carbon skeleton. The enantiomer-enriched amine ligands are represented by the general formula VII, 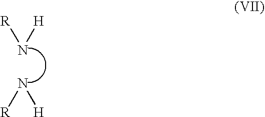
wherein particularly suitable C2-symmetrical ligands, such as are listed in “Catalytic asymmetric synthesis”, Iwao Ojima, Second Edition, Wiley-VCH 2000, may be employed.

The catalysts consisting of ligand/transition metal combinations and a corresponding diamine listed in the following Table I are particularly suitable for the enantioselective hydrogenation of the ketone (II):

TABLE 1

| Phosphorus-Containing | |
|---|---|
| Catalyst | Diamine |
| (R)-Deguphos-RuCl2 | 1,2-ethylenediamine |
| (R)-Deguphos-RuCl2 | (R,R)-DPEN |
| (R)-Deguphos-RuCl2 | (R,R)-1,2-diaminocyclohexane |
| (R)-Deguphos-RuCl2 | (R,R)-DAIPEN |
| (R)-BINAP*-RuCl2 | 1,2-ethylenediamine |
| (R)-BINAP*-RuCl2 | (R,R)-DPEN |
| (R)-BINAP*-RuCl2 | (R,R)-1,2-diaminocyclohexane |
| (R)-BINAP*-RuCl2 | (R,R)-DAIPEN |
| (S)-DIOP-RuCl2 | 1,2-ethylenediamine |
| (S)-DIOP-RuCl2 | (R,R)-DPEN |
| (S)-DIOP-RUCl2 | (R,R)-1,2-diaminocyclohexane |
| (S)-DIOP-RUCl2 | (R,R)-DAIPEN |
| (S)-PhanePHOS-RuCl2 | 1,2-ethylenediamine |
| (S)-PhanePHOS-RuCl2 | (R,R)-DPEN |
| (S)-PhanePHOS-RuCl2 | (R,R)-1,2-diaminocyclohexane |
| (S)-PhanePHOS-RuCl2 | (R,R)-DAIPEN |
| (S)-BDPP-RuCl2 | 1,2-ethylenediamine |
| (S)-BDPP-RuCl2 | (R,R)-DPEN |
| (S)-BDPP-RuCl2 | (R,R)-1,2-diaminocyclohexane |
| (S)-BDPP-RuCl2 | (R,R)-DAIPEN |
| (R)-Norphos-RuCl2 | 1,2-ethylenediamine |
| (R)-Norphos-RuCl2 | (R,R)-DPEN |
| (R)-Norphos-RuCl2 | (R,R)-1,2-diaminocyclohexane |
| (R)-Norphos-RuCl2 | (R,R)-DAIPEN |
| (S,S)-BPPM-RuCl2 | 1,2-ethylenediamine |
| (S,S)-BPPM-RuCl2 | (R,R)-DPEN |
| (S,S)-BPPM-RuCl2 | (R,R)-1,2-diaminocyclohexane |
| (S,S)-BPPM-RuCl2 | (R,R)-DAIPEN |
| (R)-ProPhos-RuCl2 | 1,2-ethylenediamine |
| (R)-ProPhos-RuCl2 | (R,R)-DPEN |
| (R)-ProPhos-RuCl2 | (R,R)-1,2-diaminocyclohexane |
| (R)-ProPhos-RuCl2 | (R,R)-DAIPEN |

*also includes TolBINAP and XylBINAP

The abbreviations of the ligand names as well as the graphic formulae of the ligands may be found in: Chemicals for Research, Catalog No. 19 from Strem, 2001-2003; Angew. Chem. 2001, 113, 40 [Lit. 16] or also in “Handbook of Chiral Chemicals”, David J. Ager, Marcel Dekker Inc., 1999.

It is known to carry out enantioselective catalytic hydrogenations by two process variants that differ in principle (with molecular hydrogen or by transfer hydrogenation). Also, the process of the subject matter of the invention may be carried out either in the presence of molecular hydrogen or by means of transfer hydrogenation. Both types of process have been evaluated in the prior art and may be used analogously (“Asymmetric transferhydrogenation of C═O and C═N bonds”, M. Wills et al. Tetrahedron: Asymmetry 1999, 10, 2045; “Asymmetric transfer hydrogenation catalysed by chiral ruthenium complexes”, R. Noyori et al. Acc. Chem. Res. 1997, 30, 97; “Asymmetric catalysis in organic synthesis”, R. Noyori, John Wiley & Sons, New York, 1994, p. 123; “Transition metals for organic Synthesis” Ed. M. Beller, C. Bolm, Wiley-VCH, Weinheim, 1998, Vol. 2, p. 97; “Comprehensive Asymmetric Catalysis” Ed.: Jacobsen, E. N.; Pfaltz, A.; Yamamoto, H., Springer-Verlag, 1999).

It has proved advantageous if a base is present in the reaction according to the invention. The use of a preferred base is governed by process technology and commercial considerations. The base should be as inexpensive as possible, but apart from this should be particularly effective and above all should not have any negative influence on for example the enantiomer purity of the products that are formed. In this connection alkali metal alcoholates are advantageous, such as for example sodium methanolate, sodium ethanolate or potassium tert.-butylate as well as potassium isopropylate or carbonates or hydroxides of alkali or alkaline earth metals. Also advantageous are organic nitrogen bases such as pyridine, DMAP, triethylamine, Hünig base, 1,2-ethylenediamine, diphenylenediamine, 1,2-di-(4-anisyl)-2-isobutyl-1,2-ethylenediamine and 1,2-di-(4-anisyl)-2-isopropyl-1,2-ethylenediamine.

It is furthermore advantageous to use these bases in a sufficient amount. It has been found that acid residues obviously affect the present reaction in that on the one hand they lead to a low yield and on the other hand cause a low enantiomer enrichment of the products. The person skilled in the art is able to determine a suitably adequate excess of base. A molar excess of base referred to the catalyst used of >1000:1 is especially advantageous, an excess of >100:1 being particularly preferred and an excess of >20:1 being most particularly preferred. One of the bases mentioned above is accordingly added to the substrate in an amount of 10-50%, particularly preferably 5-10% and most particularly preferably 1-5% referred to the latter.

All solvents known to the person skilled in the art for this purpose may be used provided that they are inert with respect to the reaction according to the invention. In particular these are alcohols, advantageously the complementary alcohols of the alcoholates listed above, such as methanol, ethanol, isopropanol, tert.-butanol in their aqueous or non-aqueous form. The use of a mixture of isopropanol and potassium tert.-butylate is most particularly preferred.

The hydrogenation catalyst comprising the diamine, transition metal and the phosphorus-containing ligand is advantageously used in a concentration of 0.01-5 mole % referred to the substrate to be hydrogenated. It is particularly preferred to use the catalyst in a concentration that is as low as possible while ensuring the optimum possible conversion rate. The catalyst is particularly preferably used in a concentration of 0.1-1 mole %, and most particularly preferably in a concentration of 0.1-0.5 mole %.

The temperature during the reaction may in principle be chosen arbitrarily by the person skilled in the art as long as a sufficiently quick and selective reaction is guaranteed. The reaction is accordingly preferably carried out at temperatures between 0° and 100° C., more preferably between 100 and 80° C. and particularly preferably between 20° 0 and 60° C.

If the hydrogenation is carried out in the presence of molecular hydrogen, then a hydrogen pressure of 1-200, preferably 2-100 and particularly preferably between 5-80 bar should be adjusted.

The present invention also provides the cyclic carbamate of the formula III.

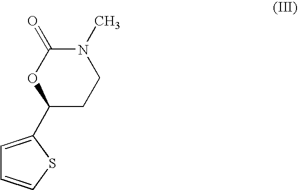

Depending on the reaction conditions, this may occur as a byproduct or main product in the hydrogenation of the corresponding carbamate-protected ketone (DE10207586), but may however advantageously be converted into the desired deprotected form by suitable hydrolysis.

In order to prepare the enantiomer-enriched N-methyl-3-(1-hydroxy)-3-(2-thienyl)propanamine the person skilled in the art proceeds by dissolving the corresponding ketone in an alcohol, adding the constituents of the hydrogenation catalyst to the mixture and then performing the hydrogenation at an appropriate temperature and suitable hydrogen pressure. Since the constituents of the hydrogenation catalyst (diamine, transition metal and phosphorus-containing ligand) may be used in several diastereomeric and enantiomeric forms and the complex formed in each case may therefore be present in so-called matched or mismatched configurations with regard to the substrate to be hydrogenated, the person skilled in the art must check which pair of enantiomer-enriched diamine and enantiomer-enriched phosphine ligand work most suitably in the hydrogenation catalyst. To prepare (S)-N-methyl-3-(1-hydroxy)-3-(2-thienyl)propanamine it has for example proved suitable to use the (S)-PhanePhos-RuCl₂-(R,R)-DPEN complex as catalyst.

(C₁-C₈)-alkyl denotes methyl, ethyl, n-propyl, isopropyl, n-butyl, isobutyl, sec-butyl, tert-butyl, pentyl, hexyl, heptyl or octyl, as well as all bond isomers.

(C₁-C₈)-alkoxy denotes a (C₁-C₈)-alkyl radical bound via an oxygen atom to the molecule in question.

(C₁-C₈)-acyl denotes a (C₁-C₈)-alkyl radical bound via a —C(═O) function to the molecule in question.

(C₁-C₈)-alkoxycarbonyl denotes a (C₁-C₈)-alkyl radical bound via a —O—C(═O) function to the molecule.

A (C₆-C₁₈)-aryl radical is understood to denote an aromatic radical with 6 to 18 C atoms. This includes in particular species such as phenyl, naphthyl, anthryl, phenanthryl and biphenyl radicals. These may be substituted singly or multiply with (C₁-C₈) -alkoxy, (C₁-C₈)-haloalkyl, OH, Cl, NH₂, NO₂. Also, the radical may contain one or more heteroatoms such as N, O, S.

A (C₇-C₁₉)-aralkyl radical is a (C₆-C₁₈)-aryl radical bound via a (C₁-C₈)-alkyl radical to the molecule.

(C₁-C₈)-haloalkyl is a (C₁-C₈)-alkyl radical substituted with one or more halogen atoms. Suitable halogen atoms are in particular chlorine and fluorine.

A (C₃-C₁₈)-heteroaryl radical denotes within the scope of the invention a five-membered, six-membered or seven-membered aromatic ring system of 3 to 18 C atoms that contains heteroatoms such as for example nitrogen, oxygen or sulfur in the ring. Such heteroaromatics are in particular radicals such as 1-, 2-, 3-furyl, 1-, 2-, 3-pyrrolyl, 1-, 2-, 3-thienyl, 2-, 3-, 4-pyridyl, 2-, 3-, 4-, 5-, 6-, 7-indolyl, 3-, 4-, 5-pyrazolyl, 2-,4-, 5-imidazolyl, acridinyl, chinolinyl, phenanthridinyl, 2-, 4-, 5-, 6-pyrimidinyl. These may be singly or multiply substituted with (C₁-C₈)-alkoxy, (C₁-C₈)-haloalkyl, OH, halogen, NH₂, NO₂, SH, S—(C₁-C₈)-alkyl.

A (C₄-C₁₉)-heteroaralkyl is understood to denote an heteroaromatic system corresponding to the (C₇-C₁₉)-aralkyl radical.

The expression (C₁-C₈)-alkylene bridge is understood to mean a (C₁-C₈)-alkyl radical that is bound via two different C atoms to the relevant molecule. This may be singly or multiply substituted with (C₁-C₈)-alkoxy, (C₁-C₈)-haloalkyl, OH, halogen, NH₂, NO₂, SH, S—(C₁-C₈)-alkyl or (C₆-C₁₈) -aryl.

(C₃-C₈)-cycloalkyl is understood to denote cyclopropyl, cyclobutyl, cyclopentyl, cyclohexyl, cycloheptyl or cyclooctyl radicals. This may be singly or multiply substituted with (C₁-C₈)-alkoxy, (C₁-C₈)-haloalkyl, OH, halogen, NH₂, NO₂, SH, S—(C₁-C₈)-alkyl or (C₆-C₁₈)-aryl.

Halogen is fluorine, chlorine, bromine or iodine.

The illustrated chemical structures relate to all possible stereoisomers that can be obtained by altering the configuration of the individual chiral centres, axes or planes, i.e. all possible diastereomers as well as all optical isomers (enantiomers) included therein.

Enantiomer-enriched or enantiomerically enriched denotes the presence in the mixture of an enantiomer in an amount of >50% compared to its optical antipode.

The specifications cited here are considered to be part of the disclosure. This application refers to the priority application DE10233724 which is herewith incorporated by referrence to its entirety. In particular it is referred to the disclosure of the usage of the Phanephos as ligand in present reaction. All the possibilities for residues R₂ or X¹ and X² mentioned in DE10233724 for compounds of formula (III) may be equally applied herein.

### EXAMPLE 1

### (S)-3-N-ethoxycarbonyl-N-methylamino-1-(2-hienyl)-1-propanol

4.9 g (20.4 mmole) of 3-N-ethoxycarbonyl-N-methylamino-1-(2-thienyl)-1-propanone are added to a 100 ml Büchi stirred autoclave and the latter is evacuated. 18.4 mg (0.02 mmole) of (R)-TolBINAP-RuCl₂-(1R, 2R)-diphenylethylene-diamine are dissolved together with 0.4 ml (0.4 mmole) of a 1 M potassium tert.-butylate solution in. 40 ml of isopropanol, stirred for 15 minutes, and sucked into the autoclave. After flushing with hydrogen, hydrogen is pumped in under a pressure of 10 bar and the mixture is hydrogenated for 2 hours at 40° C. The reaction mixture is filtered through Celite and concentrated by evaporation. 5.8 g of a yellowish-brown oil remain, which according to HPLC contains the desired alcohol in an enantiomer excess (ee) of 80.1%. The conversion is >96%. After standing for a fairly long time, the content of cyclic carbamate (III) increases significantly.

¹H-NMR (DMSO-d⁶): 1.15 (t,CH₃), 1.9 (m, CH₂), 2.85 (s, N—CH₃), 3.20 (m, CH₂), 4.0 (q, O—CH₂), 4.8 (m, CH), 5.65 (t, OH), 6.95 (m, 2H-arom.), 7.4 (m, 1H-arom.).

### EXAMPLE 2

### (S)-[(N-methyl)-4-(2-thienyl)-tetrahydro-2H-oxazin-2-one (cyclic carbamate III).

50 g (207.4 mmole) of 3-N-ethoxycarbonyl-N-methylamino-1-(2-thienyl)-1-propanone are placed in a 1 1 stirred autoclave which is then evacuated. 195 mg (0.2 mmole) of (R)-TolBINAP-RuCl₂-(1R, 2R)-diphenylethylenediamine are dissolved together with 2.2 ml (2.2 mmole) of a 1 M potassium tert.-butylate solution in 450 ml of isopropanol, stirred for 15 minutes and sucked into the autoclave. After flushing with hydrogen, hydrogen is forced in under a pressure of 10 bar and the mixture is hydrogenated for 24 hours at 40° C. The reaction mixture is filtered through Celite and concentrated by evaporation. 52 g of a yellowish-brown oil remain, which slowly solidifies on standing. According to HPLC the oil contains the desired compound in an amount of >80%. 20 g of the crude product are stirred in isopropanol and suction filtered. The raw material is recrystallised from isopropanol. 6.7 g (34%) of the cyclic carbamate were obtained.

¹H-NMR (DMSO-d⁶): 2.18 (m, CH₂), 2.85 (s, N—CH₃), 3.35 (m, CH₂), 5.6 (dd, O—CH), 7.0 (m, 1H-arom.), 7.15 (m, 1H-arom.), 7.55 (m, 1H).

### EXAMPLE 3

4.9 g (20.4 mmole) of 3-N-ethoxycarbonyl-N-methylamino-1-(2-thienyl)-1-propanone are placed in a 100 ml Büchi stirred autoclave, which is then evacuated. 4.9 mg (0.51 mmole) of (S)-PhanePhos-RuCl₂-(1R, 2R)-diphenylethylenediamine are dissolved together with 0.8 ml (0.8 mmole) of a 1 M potassium tert.-butylate solution in 40 ml of isopropanol, stirred for 15 minutes, and sucked into the autoclave. After flushing with hydrogen, hydrogen is forced in under a pressure of 10 bar and the reaction mixture is hydrogenated for 2 hours at 40° C. The reaction mixture is filtered through Celite and the filtrate is concentrated by evaporation. 4.1 g of a yellowish-brown oil remain, which according to HPLC has an ee of 93.4%.

The monomethyl alcohol can be obtained according to a known procedure, which is described in application DE10207586, in >99% ee from the enantiomer-enriched alcohol or cyclic carbamate after splitting off the protective groups.

1. A process for the preparation of enantiomer-enriched compounds of the general formula (I) 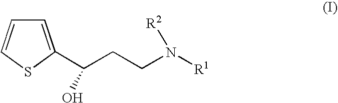
wherein
R¹ and R² independently of one another denote H, (C₁-C₈)-alkyl, (C₁-C₈)-acyl, (C₁-C₈)-alkoxycarbonyl, (C₃-C₈)-cycloalkyl, (C₆-C18)-aryl, (C₇-C₁₉)-aralkyl, (C₃-C₁₈)-heteroaryl, (C₄-C₁₉)-heteroaralkyl, ((C₁-C₈)-alkyl)₁₋₃-(C₃-C₈)-cycloalkyl, ((C₁-C₈)-alkyl)₁₋₃—(C₆-C₁₈)-aryl, ((C₁-C₈)-alkyl)₁₋₃—(C₃-C₁₈)-heteroaryl,
or the radicals R¹ and R² together form a (C₁-C₈)-alkylene bridge, wherein one or more carbons in said (C₁-C₈)-alkylene bridge may be substituted with one or more radicals selected from the group consisting of: (C₁-C₈)-alkyl, (C₃-C₈)-cycloalkyl, (C₆-C₁₈)-aryl, (C₇-C₁₉)-aralkyl, (C₃-C₁₈)-heteroaryl, (C₄-C₁₉)-heteroaralkyl with the formation of further chirality centres,
comprising enantioselectively hydrogenating compounds of the general formula (II) 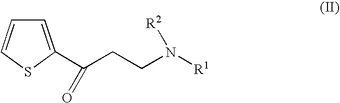
wherein R¹ and R² have the meanings given above,
with a catalyst comprising an enantiomer-enriched bidentate phosphorus-containing ligand, a transition metal and a diamine.

2. The process of claim 1, wherein said enantiomer-enriched bidentate phosphorus-containing ligand is a chiral compound selected from the group consisting of Deguphos, Binap, Phanephos, Norphos, DIOP, Duphos, Prophos, BDPP, BPPM, Malphos, Rophos and Basphos.

3. The process of claim 1, wherein said diamine is a chiral compound selected from the group consisting of DIAPEN, DPEN, DMDPEN, and 1,2-cyclohexyldiamine.

4. The process of claim 1, wherein said transition metal is selected from the group consisting of: Ru, Rh, Ir, and Pd.

5. The process of any one of claims 1-4, wherein hydrogenation is carried out in the presence of molecular hydrogen or by transfer hydrogenation.

6. The process of any one of claims 1-4, wherein hydrogenation is carried out in the presence of a base.

7. The process of claim 6, wherein said base is used in a molar ratio of at least 10 to 1 relative to said catalyst.

8. The process of any one of claims 1-4, wherein hydrogenation is carried out in a solvent selected from the group consisting of methanol, ethanol, isopropanol, and tert.-butanol, in their aqueous or non-aqueous form.

9. The process of any one of claims 1-4, wherein said catalyst comprising said diamine, transition metal and phosphorus-containing ligand is used at a concentration of 0.1-0.5 mole %.

10. The process of any one of claims 1-4, wherein hydrogenation is carried out at a temperature of between 0° and 100° C.

11-12. (canceled)

13. The process of claim 10, wherein said temperature is between 10° and 80° C.

14. The process of claim 10, wherein said temperature is between 20° and 60° C.

15. The process of any one of claims 1-4, wherein hydrogenation is carried out in the presence of molecular hydrogen at a hydrogen pressure of 1-200 bar.

16. The process of claim 15, wherein said pressure is 2-100 bar.

17. The process of claim 15, wherein said pressure is 5-80 bar.

18. The process of claim 5, wherein hydrogenation is carried out in the presence of a base.

19. The process of claim 18, wherein said base is used in a molar ratio of at least 10 to 1 relative to said catalyst.

20. The process of claim 19, wherein hydrogenation is carried out in a solvent selected from the group consisting of methanol, ethanol, isopropanol, and tert.-butanol, in their aqueous or non-aqueous form.

21. The process of claim 20, wherein said catalyst comprising said diamine, transition metal and phosphorus-containing ligand is used at a concentration of 0.1-0.5 mole %.

22. The process of claim 21, wherein hydrogenation is carried out at a temperature of between 20° C. and 60° C.

23. The process of any one of claim 22, wherein hydrogenation is carried out in the presence of molecular hydrogen at a hydrogen pressure of 5-80 bar.

24. A cyclic carbamate of formula III: [chemical structure]
